(19) United States
(12) Patent Application Publication
(10) Pub. No.: US 2020/0237192 A1
(43) Pub. Date: Jul. 30, 2020

(54) CASE BODY AND INSERTION APPARATUS

(71) Applicant: OLYMPUS CORPORATION, Tokyo (JP)

(72) Inventors: Kentaro KONO, Tokyo (JP); Kunihiko HIJIHARA, Tokyo (JP)

(73) Assignee: OLYMPUS CORPORATION, Tokyo (JP)

(21) Appl. No.: 16/847,105

(22) Filed: Apr. 13, 2020

Related U.S. Application Data
(63) Continuation of application No. PCT/JP2018/030859, filed on Aug. 21, 2018.

(30) Foreign Application Priority Data
Oct. 17, 2017 (JP) 2017-201168

(51) Int. Cl. A61B 1/00

(52) U.S. Cl. CPC A61B 1/00071

(57) ABSTRACT

A case body includes a distal end side case configuring a part of a case body exterior, a center portion case disposed in a position adjacent to the distal end side case and configuring a case body exterior in conjunction with the distal end side case, a first elastic seal member disposed to be exposed as a case body exterior in a boundary between the distal end side case and the center portion case provided adjacent to each other, and an abutting section configured to set a crushing amount of the first elastic seal member, the abutting section including an abutting surface provided in one of the distal end side case and the center portion case and coming into contact with a bottom surface of another exterior member.

### CROSS REFERENCE TO RELATED APPLICATION

This application is a continuation application of PCT/JP2018/030859 filed on Aug. 21, 2018 and claims benefit of Japanese Application No. 2017-201168 filed in Japan on Oct. 17, 2017, the entire contents of which are incorporated herein by this reference.

### BACKGROUND OF THE INVENTION

### 1. Field of the Invention

The present invention relates to a case body including exterior members adjacently provided to form an internal space and an elastic seal member disposed in a boundary between the exterior members and an insertion apparatus including the case body.

### 2. Description of the Related Art

An endoscope is used in a medical field, an industrial field, and the like. The endoscope is an insertion apparatus and generally includes an elongated insertion section. An operation section is provided on a proximal end side of the insertion section. A universal cord extends from the operation section. An endoscope connector is provided at a proximal end portion of the universal cord.

The operation section of the endoscope, the endoscope connector, or the like is a case body configuring an exterior and includes a first exterior member and a second exterior member having a tubular shape or a cylindrical shape. The case body, to which the first exterior member and the second exterior member are integrally assembled, has an internal space. In the case body, for example, an O-shaped ring, which is an elastic seal member, is disposed in a boundary between the first exterior member and the second exterior member. The elastic seal member retains watertightness between the first exterior member and the second exterior member and blocks intrusion of liquid into the internal space.

For example, Japanese Patent Application Laid-Open Publication No. 2006-130238 discloses an image pickup apparatus for endoscope including a bend preventing portion that can secure satisfactory assemblability and obtain sure waterproofness and is configured to facilitate disassembling and assembling for repair and the like.

An exterior of the image pickup apparatus for endoscope (a sign 3 in FIG. 2) shown in FIG. 2 of Japanese Patent Application Laid-Open Publication No. 2006-130238 equivalent to a case body of the present invention is configured by a front end exterior member (a sign 37 in FIG. 2), an intermediate exterior member (a sign 36 in FIG. 2), and a rear end exterior member (a sign 30 in FIG. 2).

An O-shaped ring (a sign 35 in FIG. 2) for retaining watertightness is disposed between the rear end exterior member and the intermediate exterior member. An end face on an opening side of a step section and one end face of the intermediate exterior member approaches, whereby this O-shaped ring is crushed and deformed to retain watertightness.

On the other hand, an O-shaped ring (a sign 38 in FIG. 2) is disposed between the intermediate exterior member and the front end exterior member. One end portion of the front end exterior member is inscribed with an inner circumferential surface on a front end side of the intermediate exterior member, whereby this 0-shaped ring is crushed and deformed to retain watertightness.

Japanese Patent Application Laid-Open Publication No. 2006-130238 discloses an image pickup apparatus for endoscope (a sign 3A in FIG. 3) including a front end exterior member (a sign 37A in FIG. 3), an intermediate exterior member (a sign 36A in FIG. 3), and a rear end exterior member (a sign 30A in FIG. 3). In the image pickup apparatus for endoscope, an L-shaped watertight seal member (a sign 41 in FIG. 3) is provided between the intermediate exterior member and the rear end exterior member. The rear end exterior member and a second metal frame are screwed and coupled and fixed, whereby this watertight seal member is crushed to be deformed into a state for retaining watertightness.

### SUMMARY OF THE INVENTION

A case body according to an aspect of the present invention includes: a first exterior member configuring a part of a case body exterior; a second exterior member disposed in a position adjacent to the first exterior member and configuring the case body exterior in conjunction with the first exterior member; an elastic seal member having predetermined elasticity disposed to be exposed as the case body exterior in a boundary between the first exterior member and the second exterior member provided adjacent to each other; and an abutting member configured to set a crush amount of the elastic seal member, the abutting member including an abutting surface provided in one of the first exterior member and the second exterior member and coming into contact with another exterior member. The abutting member is a projection provided in the first exterior member and projecting with respect to a first boundary surface configuring one side of the boundary. The second exterior member includes a recess formed to recess with respect to a second boundary surface configuring another side of the boundary, the projection being housed in the recess. The elastic seal member is held between the first boundary surface and the second boundary surface in a state in which the projection and the recess are engaged. The elastic seal member is formed in a stepped shape including a large diameter section formed in a ring shape and a small diameter section formed in a ring shape smaller in diameter than the large diameter section. An outer circumferential surface of the large diameter section is disposed to be exposed as the case body exterior in the boundary between the first exterior member and the second exterior member.

A case body according to another aspect of the present invention includes: a first exterior member configuring a part of a case body exterior; a second exterior member disposed in a position adjacent to the first exterior member and configuring the case body exterior in conjunction with the first exterior member; an elastic seal member having predetermined elasticity, the elastic seal member including a large diameter section formed in a ring shape and a small diameter section formed in a ring shape smaller in diameter than the large diameter section and being disposed to be exposed as the case body exterior in a boundary between the first exterior member and the second exterior member provided adjacent to each other; an abutting member configured to set a crush amount of the elastic seal member, the abutting member including an abutting surface provided in one of the first exterior member and the second exterior member and coming into contact with another exterior member; a watertightness retaining member disposed to be further separated from the case body exterior than the elastic seal member in the boundary between the first exterior member and the second exterior member and configured to retain watertightness; and a coupling ring formed to project from the small diameter section and coupled to the watertightness retaining member.

An insertion apparatus according to an aspect of the present invention includes: an insertion section configured to be inserted into a subject; and a case body including: a first exterior member provided on a distal end side of the insertion section and configuring a part of an internal space and a part of a case body exterior; a second exterior member disposed in a position adjacent to the first exterior member and configuring the case body exterior in conjunction with the first exterior member; an elastic seal member having predetermined elasticity disposed to be exposed as the case body exterior in a boundary between the first exterior member and the second exterior member provided adjacent to each other; and an abutting member configured to set a crush amount of the elastic seal member, the abutting member including an abutting surface provided in one of the first exterior member and the second exterior member and coming into contact with another exterior member, wherein the abutting member is a projection provided in the first exterior member and projecting with respect to a first boundary surface configuring one side of the boundary, the second exterior member includes a recess formed to recess with respect to a second boundary surface configuring another side of the boundary, the projection being housed in the recess, the elastic seal member is held between the first boundary surface and the second boundary surface in a state in which the projection and the recess are engaged, and the elastic seal member is formed in a stepped shape including a large diameter section formed in a ring shape and a small diameter section formed in a ring shape smaller in diameter than the large diameter section, and an outer circumferential surface of the large diameter section is disposed to be exposed as the case body exterior in the boundary between the first exterior member and the second exterior member.

An insertion apparatus according to another aspect of the present invention includes: an insertion section configured to be inserted into a subject; and a case body including: a first exterior member provided on a distal end side of the insertion section and configuring a part of an internal space and a part of a case body exterior; a second exterior member disposed in a position adjacent to the first exterior member and configuring the case body exterior in conjunction with the first exterior member; an elastic seal member having predetermined elasticity, the elastic seal member including a large diameter section formed in a ring shape and a small diameter section formed in a ring shape smaller in diameter than the large diameter section and being disposed to be exposed as the case body exterior in a boundary between the first exterior member and the second exterior member provided adjacent to each other; an abutting member configured to set a crush amount of the elastic seal member, the abutting member including an abutting surface provided in one of the first exterior member and the second exterior member and coming into contact with another exterior member; a watertightness retaining member disposed to be further separated from the case body exterior than the elastic seal member in the boundary between the first exterior member and the second exterior member and configured to retain watertightness; and a coupling ring formed to project from the small diameter section and coupled to the watertightness retaining member.

### BRIEF DESCRIPTION OF THE DRAWINGS

FIG. 1 is a diagram for explaining an endoscope, which is one of operation instruments;

FIG. 2 is a diagram for explaining a configuration example of a grasping section of the endoscope, which is a case body;

FIG. 3A is a front view of a first elastic seal member disposed in a boundary between a distal end side case and a center portion case;

FIG. 3B is an arrow Y3B-Y3B line sectional view of FIG. 3A;

FIG. 4A is a diagram for explaining a relation among the distal end side case, the first elastic seal member, and the center portion case;

FIG. 4B is a diagram for explaining a state in which the first elastic seal member is disposed in an engaging section of the distal end side case and a relation between the distal end side case and the center portion case;

FIG. 4C is a diagram for explaining the case body in which the first elastic seal member is disposed in the boundary between the distal end side case and the center portion case;

FIG. 5A is a diagram for explaining a second elastic seal member disposed in a boundary between the center portion case and a proximal end side case;

FIG. 5B is a diagram for explaining a relation among the center portion case, the second elastic seal member, and the proximal end side case;

FIG. 5C is a diagram for explaining a state in which the second elastic seal member is disposed in the boundary between the center portion case and the proximal end side case;

FIG. 6A is a diagram for explaining a two-step-shaped elastic seal member disposed in the boundary;

FIG. 6B is a diagram for explaining a relation among the distal end side case, the two-step-shaped elastic seal member, and the center portion case;

FIG. 6C is a diagram for explaining a state in which the two-step-shaped elastic seal member is disposed in the boundary between the distal end side case and the center portion case;

FIG. 7A is a diagram for explaining an elastic seal member disposed in the boundary and having a two-step-shape and including an O-shaped ring shape section;

FIG. 7B is a diagram for explaining a relation among the distal end side case, the elastic seal member having the two-step-shape and including the O-shaped ring shape section, and the center portion case;

FIG. 7C is a diagram for explaining a state in which the elastic seal member having the two-step-shape and including the O-shaped ring shape section is disposed in the boundary between the distal end side case and the center portion case;

FIG. 8A is a diagram for explaining a configuration example in which an elastic seal member is integrally provided in an exterior member;

FIG. 8B is a diagram for explaining an assembled state of the distal end side case and the center portion case in which the elastic seal member is insert-molded;

FIG. 9A is a diagram for explaining an elastic seal member including a ring shape section and the O-shaped ring shape section, a plurality of through-holes being formed in the ring shape section;

FIG. 9B is a Y9B-Y9B line sectional view of FIG. 9A;

FIG. 9C is a diagram for explaining a configuration of the center portion case and a state in which the elastic seal member is attached to the center portion case;

FIG. 9D is a diagram for explaining a relation among the distal end side case, an elastic seal member in which a plurality of through-holes are provided in a circumferential direction, and the center portion case in which a plurality of protrusion sections disposed in the through-holes are provided;

FIG. 9E is a diagram for explaining an assembled state of the distal end side case and the center portion case;

FIG. 10A is a diagram for explaining a state in which the distal end side case and the center portion case are integrally fixed using a fastening member;

FIG. 10B is a diagram for explaining the state in which the distal end side case and the center portion case are integrally fixed using the fastening member;

FIG. 10C is a diagram showing a state in which a center axis of a bolt, which is the fastening member, positionally deviates with respect to a center of figure;

FIG. 10D is a diagram showing a state in which the center axis of the bolt, which is the fastening member, and the center of figure coincide; and

FIG. 10E is a diagram for explaining a state in which the distal end side case and the center portion case are integrally fixed using the fastening member.

### DETAILED DESCRIPTION OF THE PREFERRED EMBODIMENT

An embodiment of the present invention is explained below with reference to the drawings.

Note that, in the figures used for the following explanation, a scale is sometimes differentiated for each of components in order to show the components in recognizable sizes on the drawings. In other words, the present invention is not limited only to quantities of the components, shapes of the components, ratios of sizes of the components, and relative positional relations among the components described in these figures.

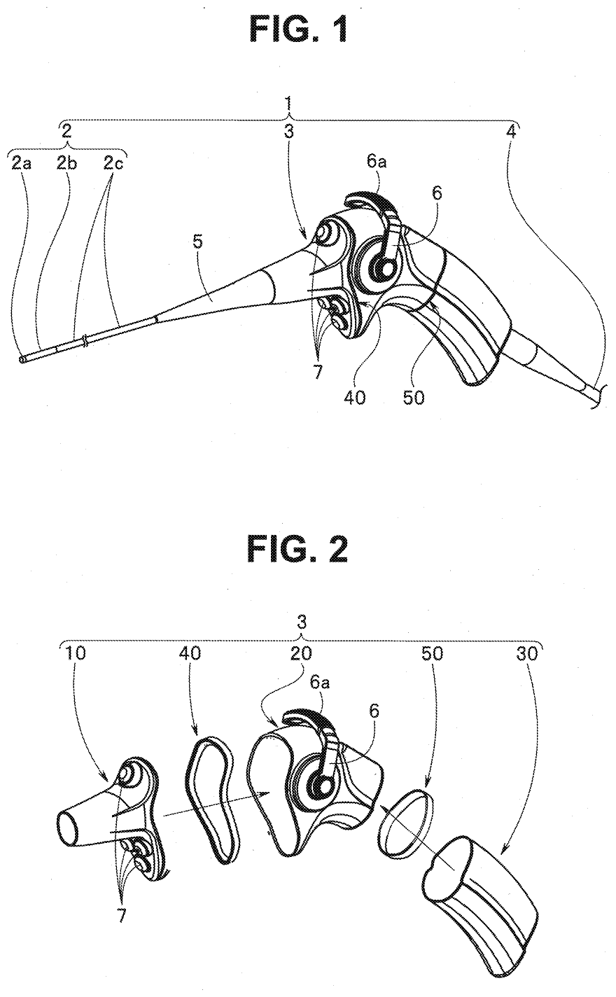

As shown in FIG. 1, a main part of an endoscope 1 is configured including an insertion section 2, a grasping section 3, and a universal cord 4. An endoscope connector (not illustrated) including a case body is disposed at an extension end portion of the universal cord 4. The endoscope 1 is an insertion apparatus including the insertion section 2 configured to be inserted into a subject.

The insertion section 2 is elongated and has flexibility. In the insertion section 2, a distal end portion 2a, a bending section 2b, and a flexible tube section 2c are concatenated in order from a distal end side. The bending section 2b is configured to be bendable in opposed two directions, for example, an upward direction and a downward direction. Note that the bending section 2b may be configured to be bendable in four directions to bend in left and right directions including a left direction and a right direction in addition to the upward and downward directions.

The grasping section 3 is coupled to, via a bend preventing portion 5, the flexible tube section 2c configuring a proximal end side of the insertion section 2. A bending operation lever 6 for performing bending operation for the bending section 2b and an operation button group 7 for executing various endoscopic functions are provided in the grasping section 3. The bending operation lever 6 is turnably pivotally supported by the grasping section 3. A finger contact section 6a is integrally fixed at an end portion projecting in a grasping section outward direction of the bending operation lever 6.

In the present embodiment, the grasping section 3 has, for example, a pistol shape. A user or the like can grasp the grasping section 3 in the same manner with either a left hand or a right hand. Therefore, the bending operation lever 6 and the operation button group 7 can be operated in the same manner with either the left hand or the right hand that grasps the grasping section 3.

The grasping section 3 is explained.

The grasping section 3 is a case body. A case body exterior of the case body is configured by a distal end side case 10, a center portion case 20, and a proximal end side case 30, which are exterior members shown in FIG. 2, and a first elastic seal member 40 and a second elastic seal member 50, which are elastic seal members. The first elastic seal member 40 and the second elastic seal member 50 have predetermined elasticity.

The distal end side case 10, the center portion case 20, and the proximal end side case 30 are rigid members and are made of metal or resin. On the other hand, the first elastic seal member 40 and the second elastic seal member 50 include materials having predetermined rubber hardness.

As shown in FIG. 1, the first elastic seal member 40 is disposed in a boundary between the distal end side case 10 and the center portion case 20 provided adjacent to each other. One surface of the first elastic seal member 40 is exposed in the boundary to configure a case body exterior.

On the other hand, the second elastic seal member 50 is disposed in a boundary between the center portion case 20 and the proximal end side case 30 provided adjacent to each other. One surface of the second elastic seal member 50 is exposed in the boundary to configure a case body exterior.

First, the first elastic seal member 40 is explained.

As shown in FIG. 2, the first elastic seal member 40 is formed in a predetermined shape.

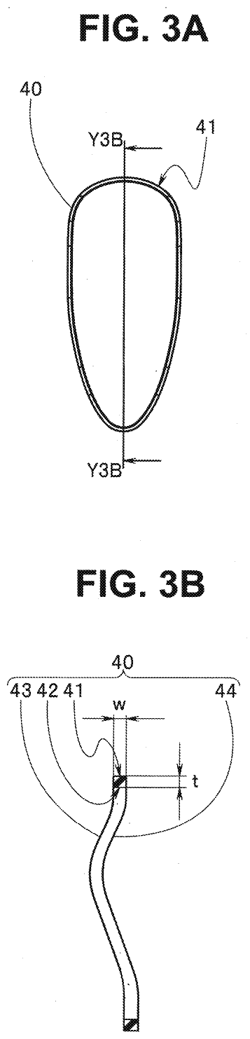

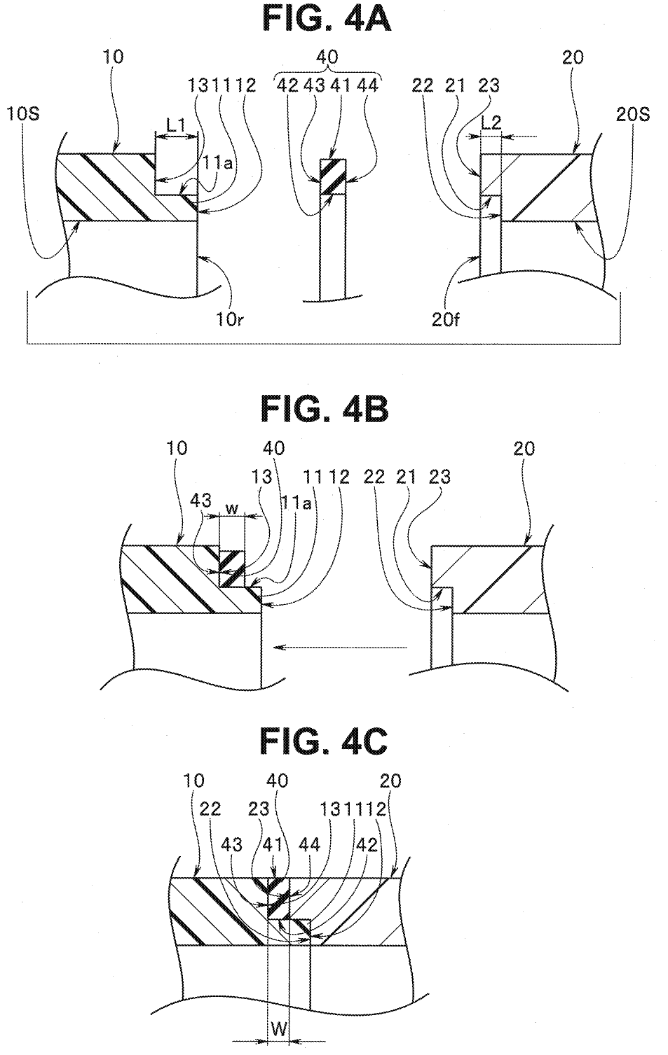

The first elastic seal member 40 has a ring shape section as shown in FIG. 3A and FIG. 3B. The first elastic seal member 40 is formed to adhere to, in a predetermined state, an outer circumferential surface of an abutting section (see a sign 11 in FIG. 4A) functioning as an abutting member, which is an engaging projection provided in a proximal end side opening of the distal end side case 10. The first elastic seal member 40 has a rectangular shape in a direction substantially orthogonal to a boundary surface explained below and is set to predetermined thickness t and predetermined width w.

A sign 41 indicates an outer circumferential surface and indicates one surface functioning as a case body exterior in a boundary. A sign 42 indicates an inner circumferential surface, a sign 43 indicates a first exterior member boundary surface contact surface (hereinafter abbreviated as a first seal first contact surface), and a sign 44 indicates a second exterior member boundary surface contact surface (hereinafter abbreviated as first seal second contact surface).

The inner circumferential surface 42 is disposed on an abutting section outer circumferential surface 11a explained below of the abutting section 11. The first seal first contact surface 43 is formed in a surface shape substantially coinciding with a surface shape of a first exterior member boundary surface 13 explained below. The first seal second contact surface 44 is formed in a surface shape substantially coinciding with a surface shape of a second exterior member distal end side boundary surface (hereinafter abbreviated as distal end side boundary surface) 23 explained below.

As shown in FIG. 4A, the first elastic seal member 40 is disposed between the distal end side case 10 configured as a first exterior member and the center portion case 20 configured as a second exterior member. A sign 10S indicates a first space section, which configures a part of an internal space of the grasping section 3. A sign 20S indicates a second space section, which configures a part of the internal space of the grasping section 3 different from the first space section.

The abutting section 11, which is a projection, is provided on a proximal end opening 10r side of the distal end side case 10. An abutting surface 12 of the abutting section 11 projects from a first exterior member boundary surface 13, which is a first boundary surface, by a predetermined distance L1. The abutting section 11 is used as both of a seal member engaging section and a center portion case engaging section. The inner circumferential surface 42 of the first elastic seal member 40 is disposed to adhere to the abutting section outer circumferential surface 11a. An inner circumferential surface of a distal end side recess (see a sign 21) of the center portion case 20 is disposed to engage in the abutting section outer circumferential surface 11a.

An inner diameter of the first elastic seal member 40 is set smaller than an outer diameter of the abutting section 11 by a predetermined dimension. An outer diameter of the first elastic seal member 40 is set smaller than an outer diameter of the distal end side case 10 by a predetermined dimension.

A housing section 21, which is a recess in which the abutting section 11 is housed, is provided on a distal end opening 20f side of the center portion case 20. A bottom surface 22 of the housing section 21 is formed to recess from the distal end side boundary surface 23, which is a second boundary surface, by a predetermined second distance L2. The bottom surface 22 is a contact surface with which the abutting surface 12 of the abutting section 11 is in contact.

In a state in which the abutting section 11 is housed in the housing section 21 and the abutting surface 12 is in contact with the bottom surface 22, a distance between the first exterior member boundary surface 13 and the distal end side boundary surface 23, which is width W of a boundary, is L1-L2. The distance between the first exterior member boundary surface 13 and the distal end side boundary surface 23 is set smaller than width w of the first elastic seal member 40.

In other words, the distance and the width w are set to a relation of L1-L2<w.

Therefore, the first elastic seal member 40 having the width w is set such that the distance between the first exterior member boundary surface 13 and the distal end side boundary surface 23 is L1-L2 and the first elastic seal member 40 is crushed by a predetermined crush amount and elastically deformed in a state in which the abutting surface 12 is in contact with the bottom surface 22.

Note that the housing section 21 may not be provided and the abutting section 11 may be configured to abut the distal end side boundary surface 23 to crush the first elastic seal member 40 by the predetermined amount and elastically deform the first elastic seal member 40.

Assembly of the distal end side case 10 and the center portion case 20 is explained.

When the distal end side case 10 and the center portion case 20 are assembled, an operator disposes the first elastic seal member 40 on the first exterior member boundary surface 13 side of the abutting section 11 in advance as shown in FIG. 4B. At that time, the operator performs fine adjustment to cause the first seal first contact surface 43 of the first elastic seal member 40 to substantially adhere to the first exterior member boundary surface 13.

Thereafter, the operator attaches the center portion case 20 to the distal end side case 10 in which the first elastic seal member 40 is disposed. In other words, the operator houses the abutting section 11 in the housing section 21, brings the abutting surface 12 and the bottom surface 22 close to each other, and moves the abutting surface 12 and the bottom surface 22 in a direction in which the first elastic seal member 40 is held between the distal end side case 10 and the center portion case 20.

Then, the distal end side boundary surface 23 comes into contact with the second boundary surface contact surface 44 located on a side of the first elastic seal member 40 opposite to the first seal first contact surface 43. Thereafter, the operator continues attachment work and brings the abutting surface 12 and the bottom surface 22 into contact with each other as shown in FIG. 4C.

As a result, the assembly of the distal end side case 10 and the center portion case 20 is completed.

The width W of the boundary in a state in which the abutting surface 12 is in contact with the bottom surface 22 is set smaller compared with the width w of the first elastic seal member 40 by a predetermined dimension. Accordingly, the first elastic seal member 40 is set to be pressed and held and crushed by the predetermined amount by the first exterior member boundary surface 13 and the distal end side boundary surface 23 and disposed without forming a gap between the first exterior member boundary surface 13 and the distal end side boundary surface 23 in an assembly completed state of the distal end side case 10 and the center portion case 20.

Therefore, watertightness in the boundary between the distal end side case 10 and the center portion case 20 can be surely retained by the first elastic seal member 40.

In a disposition state explained above, an outer circumferential surface 41 of the first elastic seal member 40 crushed by the first exterior member boundary surface 13 and the distal end side boundary surface 23 does not further project than an outer circumferential surface of the distal end side case 10 and an outer circumferential surface of the center portion case 20 and is flush or substantially flush with the outer circumferential surfaces.

Therefore, it is possible to eliminate a deficiency that, for example, a hand or fingers grasping the grasping section 3 touches the first elastic seal member 40 to adversely affect operability and applies a load to the first elastic seal member 40.

The first elastic seal member 40 is disposed being crushed by the predetermined amount without forming a gap and without the outer circumferential surface 41 further recessing than the outer circumferential surface of the distal end side case 10 and the outer circumferential surface of the center portion case 20. Accordingly, it is possible to eliminate a deficiency that filth and the like from being accumulated in a recess. It is possible to eliminate a deficiency that, because durability of the first elastic seal member 40 is deteriorated, the first elastic seal member 40 is crushed by the predetermined amount.

Next, the second elastic seal member 50 is explained.

The second elastic seal member 50 has a ring shape section and is disposed in the boundary between the center portion case 20 and the proximal end side case 30 as shown in FIG. 2.

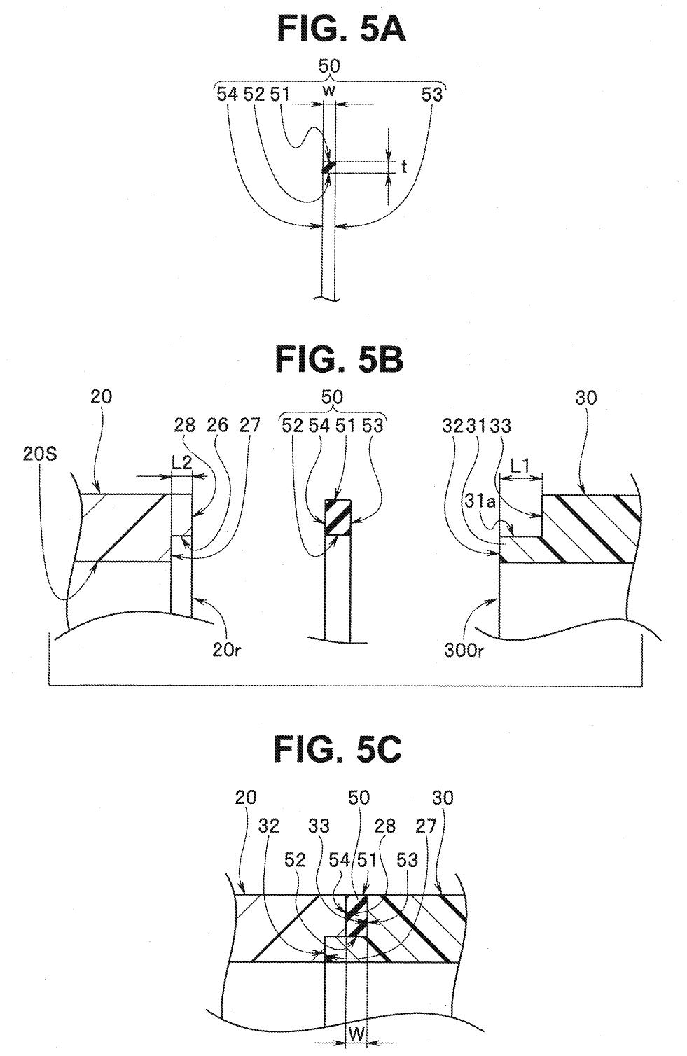

The second elastic seal member 50 shown in FIG. 5A is formed in a ring shape to adhere to, in a predetermined state, an outer circumferential surface of an abutting section 31 shown in FIG. 5B provided in a distal end side opening of the proximal end side case 30. The other components are the same as the components of the first elastic seal member 40. The second elastic seal member 50 is set to the same thickness t and the same width w as the thickness and the width of the first elastic seal member 40.

A sign 51 indicates an outer circumferential surface, which is one surface functioning as a case body exterior in a boundary. A sign 52 indicates an inner circumferential surface, a sign 53 indicates a second seal first contact surface, and a sign 54 indicates a second seal second contact surface.

The inner circumferential surface 52 is disposed on an abutting section outer circumferential surface 31a explained below of the abutting section 31. The second seal first contact surface 53 is formed in a surface shape substantially coinciding with a surface shape of a third exterior member boundary surface 33 explained below. The second seal second contact surface 54 is formed in a surface shape substantially coinciding with a surface shape of a proximal end side boundary surface 28 explained below.

Configurations of the center portion case 20 and the proximal end side case 30 are explained with reference to FIG. 5B.

As shown in FIG. 5B, the second elastic seal member 50 is disposed between the proximal end side case 30 functioning as a first exterior member and the center portion case 20 functioning as a second exterior member. A sign 30S indicates a third space section, which is an internal space different from the second space section 20S and the first space section 10S configuring a part of the internal space of the grasping section 3.

The abutting section 31, which is the projection, is provided on a distal end opening 30f side of the proximal end side case 30. An abutting surface 32 of the abutting section 31 projects from a third exterior member boundary surface 33, which is a first boundary surface, by the predetermined first distance L1. Note that the second seal first contact surface 53 is formed in a surface shape substantially coinciding with a surface shape of the third exterior member boundary surface 33.

The abutting section 31 is used as both of a seal member engaging section and a center portion case engaging section. The inner circumferential surface 52 of the second elastic seal member 50 is disposed to adhere to the abutting section outer circumferential surface 31a and an inner circumferential surface of the center portion case 20 is disposed to engage in the abutting section outer circumferential surface 31a.

An inner diameter of the second elastic seal member 50 is set, substantially the same as the inner diameter of the first elastic seal member 40, smaller than an outer diameter of the abutting section 31 by a predetermined dimension. An outer diameter of the second elastic seal member 50 is set smaller than an outer diameter of the proximal end side case 30 by a predetermined dimension.

A housing section 26, which is a recess in which the abutting section 31 is housed, is provided on a proximal end side opening 20r side of the center portion case 20. A bottom surface 27 of the housing section 26 is formed to recess from a second exterior member proximal end side boundary surface (hereinafter abbreviated as proximal end side boundary surface) 28, which is a second boundary surface, by a predetermined second distance L2. Note that the second seal second contact surface 54 is formed in a surface shape substantially coinciding with the surface shape of the proximal end side boundary surface 28.

The bottom surface 27 is a contact surface with which the abutting surface 32 of the abutting section 31 is in contact. In a state in which the abutting section 31 is housed in the housing section 26 and the abutting surface 32 is in contact with the bottom surface 27, a distance between the proximal end side boundary surface 28 and the third exterior member boundary surface 33, which is the width W of the boundary, is set smaller than the width w of the second elastic seal member 50.

In other words, the distance and the width w are a relation of L1-L2<w.

Therefore, the second elastic seal member 50 having the width w is set such that the distance between the proximal end side boundary surface 28 and the third exterior member boundary surface 33 is L1-L2 and the second elastic seal member 50 is crushed by a predetermined crush amount and elastically deformed in a state in which the abutting surface 32 is in contact with the bottom surface 27.

Assembly of the center portion case 20 and the proximal end side case 30 is explained with reference to FIG. 5B and FIG. 5C.

When assembling the center portion case 20 and the proximal end side case 30, the operator disposes the second elastic seal member 50 on the third exterior member boundary surface 33 side of the abutting section 31 in advance as shown in FIG. 5B. At that time, the operator causes the second seal first contact surface 53 of the second elastic seal member 50 to substantially adhere to the third exterior member boundary surface 33.

Thereafter, the operator attaches the proximal end side case 30, in which the second elastic seal member 50 is disposed, to the center portion case 20. In other words, the operator houses the abutting section 31 in the housing section 26, brings the abutting surface 32 and the bottom surface 27 close to each other, and moves the abutting surface 32 and the bottom surface 27 in a direction in which the second elastic seal member 50 is held between the center portion case 20 and the proximal end side case 30.

Then, the third exterior member boundary surface 33 comes into contact with the second seal first contact surface 53 of the second elastic seal member 50. Thereafter, the operator continues the attachment work and brings the abutting surface 32 and the bottom surface 27 into contact with each other as shown in FIG. 5C.

As a result, the assembly of the center portion case 20 and the proximal end side case 30 is completed.

The width W of the boundary in a state in which the abutting surface 32 is in contact with the bottom surface 27 is set smaller compared with the width w of the second elastic seal member 50 by a predetermined dimension.

Accordingly, like the first elastic seal member 40, in an assembly completed state of the center portion case 20 and the proximal end side case 30, the second elastic seal member 50 is pressed and held and crushed by a predetermined amount by the proximal end side boundary surface 28 and the third exterior member boundary surface 33 and is disposed between the proximal end side boundary surface 28 and the third exterior member boundary surface 33 without forming a gap.

Therefore, watertightness in the boundary between the center portion case 20 and the proximal end side case 30 can be surely retained by the second elastic seal member 50.

In a disposition state explained above, the outer circumferential surface 51 of the second elastic seal member 50 crushed by the proximal end side boundary surface 28 and the third exterior member boundary surface 33 does not further project than the outer circumferential surface of the center portion case 20 and an outer circumferential surface of the proximal end side case 30 and is flush or substantially flush with the outer circumferential surfaces.

Therefore, as explained above, it is possible to eliminate a deficiency that, for example, a hand or fingers grasping the grasping section 3 touches the second elastic seal member 50 to adversely affect operability and applies a load to the second elastic seal member 50.

It is possible to eliminate a deficiency that, because durability of the second elastic seal member 50 is deteriorated, the second elastic seal member 50 is crushed by the predetermined amount.

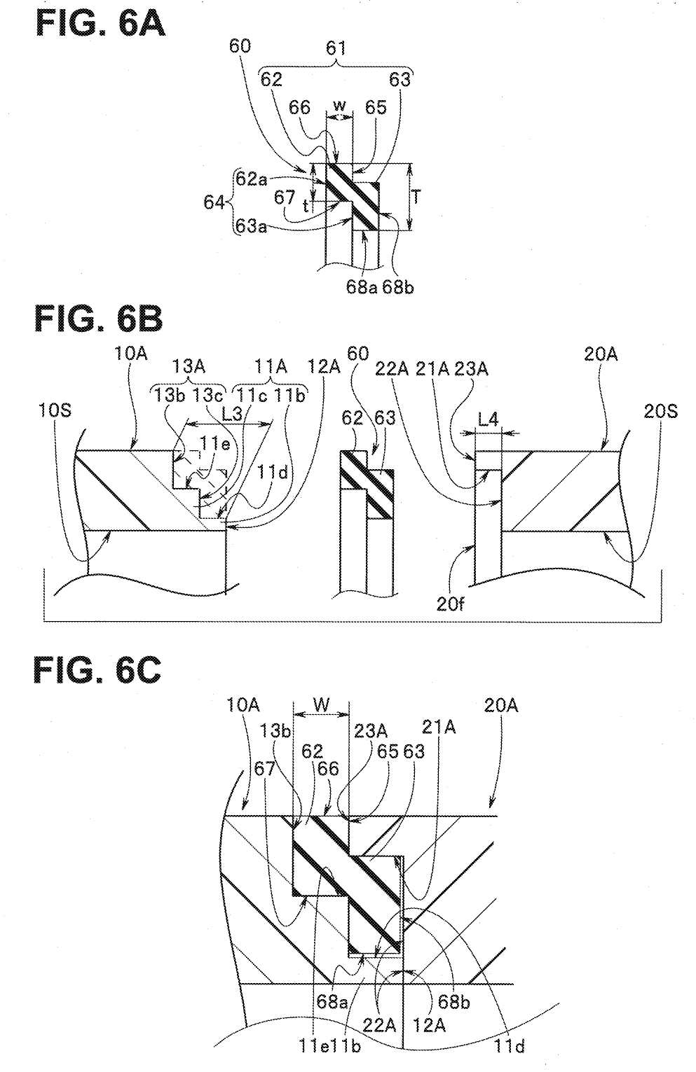

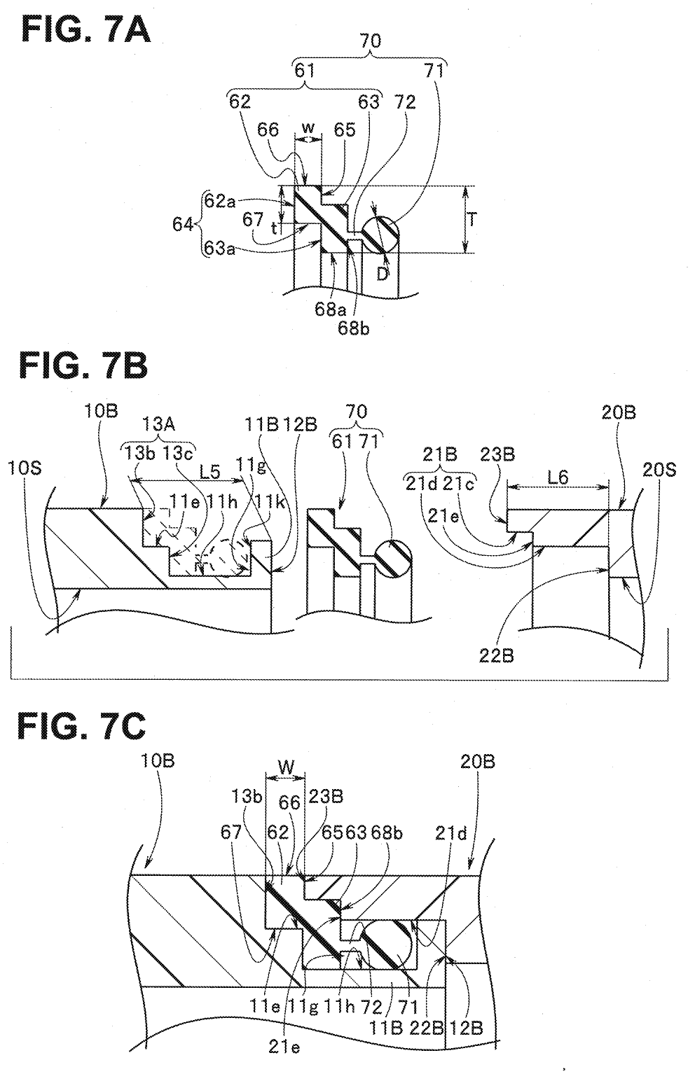

An elastic seal member 60 shown in FIG. 6A and the like and an elastic seal member 70 shown in FIG. 7A and the like, which are modifications of the elastic seal member, are explained. More specifically, the elastic seal member 60 shown in FIG. 6A has a stepped shape and has a two-step shape. The elastic seal member 70 shown in FIG. 7A includes an O-shaped ring shape section 71 in addition to the two-step shape.

First, the elastic seal member 60 is explained.

The elastic seal member 60 shown in FIG. 6A has the two-step shape, includes a step section 61, and is formed in a ring shape. The step section 61 is configured by a large diameter section 62 formed in a ring shape and a small diameter section 63 formed in a ring shape smaller in diameter than the large diameter section 62. The large diameter section 62 has thickness t and width w. Thickness and width of the small diameter section 63 is set to predetermined dimensions.

The elastic seal member 60 is disposed in a predetermined state as indicated by a broken line on an outer circumferential surface side of a stepped abutting section (hereinafter abbreviated as abutting section) 11A, which is a stepped engaging section of a distal end side case 10A, which is an exterior member shown in FIG. 6B.

The abutting section 11A includes a first projection 11b, which is a small diameter projection, and a second projection 11c, which is a large diameter projection larger in diameter than the first projection 11b, configuring a stepped convex shape section. A sign 11d indicates a first projection outer circumferential surface. A sign 11e indicates a second projection outer circumferential surface. A sign 12A indicates an abutting surface of the abutting section 11A. A sign 13A indicates a first exterior member stepped boundary surface. The first exterior member stepped boundary surface 13A includes a first outer side boundary surface 13b and a first inner side boundary surface 13c, which form a first boundary surface. The abutting surface 12A projects from the first outer side boundary surface 13b by a predetermined third distance L3.

A center portion case 20A, which is an exterior member, includes a housing section 21A. The housing section 21A is a recess that houses the first projection 11b and the small diameter section 63 of the elastic seal member 60. A bottom surface 22A of the housing section 21A is formed to recess from a distal end side boundary surface 23A, which is a second boundary surface, by a predetermined fourth distance L4. The bottom surface 22A is a contact surface with which the abutting surface 12A is in contact.

In a state in which the abutting section 11A is housed in the housing section 21A and the abutting surface 12A is in contact with the bottom surface 22A, a distance between the first outer side boundary surface 13b and the distal end side boundary surface 23A, which is the width W of the boundary, is L3-L4. The distance between the first outer side boundary surface 13b and the distal end side boundary surface 23A is set smaller than the width w of the large diameter section 62 of the elastic seal member 60.

In other words, the distance and the width w are set in a relation of L3-L4<w.

Therefore, in a state in which the abutting surface 12A is in contact with the bottom surface 22A, the large diameter section 62 having the width w is set such that the distance between the first outer side boundary surface 13b and the distal end side boundary surface 23A is L3-L4 and the large diameter section 62 is crushed by a predetermined amount and elastically deformed.

Note that width obtained by combining the large diameter section 62 having the width w and the small diameter section 63 is set smaller than L3. A sign 64 shown in FIG. 6A indicates a first contact surface. A sign 65 shown in FIG. 6A indicates a second contact surface. The first contact surface 64 includes a large diameter section contact surface 62a and a small diameter section contact surface 63a. The first contact surface 64 is formed in a surface shape substantially coinciding with a surface shape of the first exterior member stepped boundary surface 13A of the distal end side case 10A. The second contact surface 65 is formed in a surface shape substantially coinciding with a surface shape of the distal end side boundary surface 23A of the center portion case 20A.

A sign 66 shown in FIG. 6A indicates a large diameter section outer circumferential surface and is one surface functioning as a case body exterior in a boundary. A sign 67 indicates a large diameter section inner surface and is an inner circumferential surface of the large diameter section 62. An inner diameter of the large diameter section inner surface 67 is smaller than an outer diameter of the second projection outer circumferential surface 11e. Therefore, the large diameter section inner surface 67 is disposed to adhere to the second projection outer circumferential surface 11e.

Signs 68a and 68b in FIG. 6A indicate relief surfaces. An inner diameter of a first relief surface 68a is larger than an outer diameter of the first projection outer circumferential surface 11d. Therefore, the first relief surface 68a is disposed to form a predetermined gap with respect to the first projection outer circumferential surface 11d.

In other words, in a state in which the elastic seal member 60 is disposed in the abutting section 11A of the distal end side case 10A, the large diameter section inner surface 67 adheres to the second projection outer circumferential surface 11e and a gap is formed between the first projection outer circumferential surface 11d and the first relief surface 68a, which is a small diameter section inner circumferential surface.

Width obtained by combining the large diameter section 62 having the width w and the small diameter section 63 is set smaller than L3. Therefore, in a state in which the abutting surface 12A is in contact with the bottom surface 22A, the second relief surface 68b of the elastic seal member 60 is disposed to form a predetermined gap with respect to the bottom surface 22A.

The same members as the members in the embodiment explained above are indicated by the same signs and explanation of the members is omitted.

Assembly of the distal end side case 10A and the center portion case 20A and a relation of the elastic seal member 60 are explained with reference to FIG. 6B and FIG. 6C.

As shown in FIG. 6B, the elastic seal member 60 is disposed between the distal end side case 10A configured as a first exterior member and the center portion case 20A configured as a second exterior member. More specifically, the large diameter section inner surface 67 adheres to the second projection outer circumferential surface 11e and the elastic seal member 60 is disposed in the abutting section 11A as indicated by a broken line.

The small diameter section 63 of the elastic seal member 60 disposed in the abutting section 11A and the first projection 11b of the abutting section 11A are engaged in the housing section 21A of the center portion case 20A and moved in a direction in which the elastic seal member 60 is held between the distal end side case 10A and the center portion case 20A.

Then, as shown in FIG. 6C, the distal end side boundary surface 23A comes into contact with the second contact surface 65 of the elastic seal member 60. Thereafter, the bottom surface 22A does not come into contact with the second relief surface 68b, the abutting surface 12A comes into contact with the bottom surface 22A, the small diameter section 63 and the first projection 11b are housed in the housing section 21A in a predetermined state, and the assembly of the distal end side case 10A and the center portion case 20A is completed.

In this way, in an assembly completed state, the large diameter section 62 having the width w is pressed and held and crushed by a predetermined amount by the first outer side boundary surface 13b and the distal end side boundary surface 23A and is disposed between the first outer side boundary surface 13b and the distal end side boundary surface 23A without forming a gap. Therefore, watertightness in a boundary between the distal end side case 10A and the center portion case 20A can be surely retained by the large diameter section 62 of the elastic seal member 60.

In this assembly completed state, the large diameter section outer circumferential surface 66 does not further project than an outer circumferential surface of the distal end side case 10A and an outer circumferential surface of the center portion case 20A and is flush or substantially flush with the outer circumferential surfaces. As a result, as explained above, it is possible to eliminate a deficiency that a hand or fingers grasping the grasping section 3 touches the elastic seal member 60 to adversely affect operability.

In addition, the large diameter section inner surface 67 adheres to the second projection outer circumferential surface 11e in a predetermined state. Note that the first relief surface 68a of the small diameter section 63 is disposed to form a gap with respect to or to be close to the first projection outer circumferential surface 11d. The second relief surface 68b is disposed to form a gap with respect to or to be close to the bottom surface 22A.

As a result, when a finger touches the large diameter section outer circumferential surface 66 of the elastic seal member 60 and an external force is applied to the large diameter section outer circumferential surface 66, the elastic seal member 60 is elastically deformed and the large diameter section inner surface 67 is in an abutted state with the second projection outer circumferential surface 11e. The large diameter section outer circumferential surface 66 is in a state in which the large diameter section outer circumferential surface 66 is not further pushed in by the external force.

Thereafter, when the finger is released from the large diameter section outer circumferential surface 66, the large diameter section inner surface 67 in a state of being elastically deformed and abutting the second projection outer circumferential surface 11e returns to an original state of the large diameter section inner surface 67. The large diameter section outer circumferential surface 66 returns to an original state of the large diameter section outer circumferential surface 66 without recessing with the external force.

As a result, it is possible to eliminate a deficiency that an outer circumferential surface is deformed by an applied external force. Other action and effects are the same as those in the embodiment explained above.

Next, the elastic seal member 70 is explained.

The elastic seal member 70 shown in FIG. 7A includes the O-shaped ring shape section 71 functioning as a watertightness retaining member, a sectional shape of which is a substantially circular shape, in a position separated from the second relief surface 68b of the small diameter section 63 of the step section 61 formed in a ring shape of the elastic seal member 60 shown in FIG. 6A. More specifically, the O-shaped ring shape section 71 is integrally provided via a connecting section 72, which is a coupling ring formed to project from the second relief surface 68b of the small diameter section 63.

Note that the O-shaped ring shape section 71 has a diameter D. The connecting section 72 is smaller in diameter than a diameter of the O-shaped ring shape section 71.

The elastic seal member 70 including the O-shaped ring shape section 71 is disposed in a predetermined state as indicated by a broken line on an outer circumferential surface side of a stepped abutting section (hereinafter abbreviate as abutting section) 11B, which is a stepped engaging section of a distal end side case 10B, which is an exterior member, as shown in FIG. 7B.

An abutting section 11B in the present embodiment includes, in a halfway part, a circumferential groove 11g having a stepped shape. The small diameter section 63, the connecting section 72, and the O-shaped ring shape section 71 are disposed in the circumferential groove 11g. A sign 11h indicates a circumferential groove bottom surface. Depth d of the circumferential groove 11g is set smaller than the diameter D of the O-shaped ring shape section 71 by a predetermined dimension.

In other words, the depth d and the diameter D are in a relation of d<D.

Therefore, in a state in which an abutting surface 12B is in contact with a bottom surface 22B, the O-shaped ring shape section 71 is crushed by a predetermined amount and elastically deformed and adheres to the circumferential groove bottom surface 11h and an inner circumferential surface of a proximal end side recess 21d explained below and can secure watertightness.

A sign 12B indicates an abutting surface of the abutting section 11B. A sign 13A indicates a first exterior member stepped boundary surface, which includes the first outer side boundary surface 13b and the first inner side boundary surface 13c explained above. The abutting surface 12B projects from the first outer side boundary surface 13b by a predetermined fifth distance L5.

Note that the first inner side boundary surface 13c is a circumferential groove distal end side side surface of the circumferential groove 11g. Therefore, the first inner side boundary surface 13c and a circumferential groove proximal end side side surface 11k are opposed. In a state in which the small diameter section 63, the connecting section 72, and the O-shaped ring shape section 71 are disposed in the circumferential groove 11g, a predetermined gap is formed between the circumferential groove proximal end side side surface 11k and the O-shaped ring shape section 71.

A center portion case 20B, which is an exterior member, includes a stepped housing section 21B having a stepped shape. The stepped housing section 21B includes a distal end side recess 21c and a proximal end side recess 21d smaller in diameter than the distal end side recess 21c.

The small diameter section 63 of the elastic seal member 70 is housed in the distal end side recess 21c. The connecting section 72, the O-shaped ring shape section 71, and a distal end side portion of the abutting section 11B are housed in the proximal end side recess 21d. A sign 21e indicates a pressing surface. The bottom surface 22B of the proximal end side recess 21d is formed to recess from a distal end side boundary surface 23B, which is a second boundary surface, by a predetermined sixth distance L6. The bottom surface 22B is a contact surface with which the abutting surface 12B is in contact.

In a state in which the abutting section 11B is housed in the proximal end side recess 21d and the abutting surface 12B is in contact with the bottom surface 22B, a distance between the first outer side boundary surface 13b and the distal end side boundary surface 23B, which is the width W of the boundary, is L5-L6. The distance between the first outer side boundary surface 13b and the distal end side boundary surface 23B is set smaller than the width w of the large diameter section 62 of the elastic seal member 60.

In other words, the distance and the width w are set in a relation of L5-L6<w.

Therefore, the large diameter section 62 having the width w is set such that the distance between the first exterior member boundary surface 13 and the distal end side boundary surface 23B is L5-L6 and the large diameter section 62 is crushed by a predetermined crush amount and elastically deformed and adheres to at least prevent a gap from being formed in a state in which the abutting surface 12B is in contact with the bottom surface 22B.

Note that, in the present embodiment, in the state in which the abutting surface 12B is in contact with the bottom surface 22B, the second relief surface 68b is disposed to form a gap with respect to or to be close to the pressing surface 21e.

The same members as the members in the embodiment explained above are indicated by the same signs and explanation of the members is omitted.

Assembly of the distal end side case 10B and the center portion case 20B and a relation of the elastic seal member 70 are explained with reference to FIG. 7B and FIG. 7C.

As shown in FIG. 7B, the elastic seal member 70 is disposed between the distal end side case 10B configured as a first exterior member and the center portion case 20B configured as a second exterior member. More specifically, the step section 61, the O-shaped ring shape section 71, and the like of the elastic seal member 70 are disposed in the circumferential groove 11g or the like provided in the abutting section 11B as indicated by a broken line.

The abutting section 11B of the distal end side case 10B, in which the elastic seal member 70 is disposed, is engaged in the proximal end side recess 21d of the stepped housing section 21B of the center portion case 20B and moved in a direction in which the elastic seal member 70 is held between the distal end side case 10B and the center portion case 20B. Then, an inner circumferential surface of the proximal end side recess 21d of the stepped housing section 21B comes into contact with an outer side surface of the O-shaped ring shape section 71. The distal end side boundary surface 23B proceeds toward the second contact surface 65 while crushing the O-shaped ring shape section 71.

Thereafter, as shown in FIG. 7C, the distal end side boundary surface 23B comes into contact with the second contact surface 65, the pressing surface 21e comes into contact with the second relief surface 68b, and the connecting section 72, the O-shaped ring shape section 71, and the distal end side portion of the abutting section 11B are housed in the proximal end side recess 21d in a predetermined state. The assembly of the distal end side case 10B and the center portion case 20B is completed.

In this way, in an assembly completed state, the O-shaped ring shape section 71 is elastically deformed and disposed to adhere to the inner surface of the proximal end side recess 21d and the circumferential groove bottom surface 11h of the circumferential groove 11g and can surely retain watertightness in a boundary between the distal end side case 10B and the center portion case 20B.

The large diameter section 62 having the width w is pressed and held and crushed by a predetermined amount by the first outer side boundary surface 13b and the distal end side boundary surface 23B and is disposed between the first outer side boundary surface 13b and the distal end side boundary surface 23B without forming a gap. The large diameter section outer circumferential surface 66 does not further project than an outer circumferential surface of the distal end side case 10B and an outer circumferential surface of the center portion case 20B and is flush or substantially flush with the outer circumferential surfaces.

Therefore, in the assembly completed state, watertightness of the distal end side case 10B and the center portion case 20B can be more surely secured by the O-shaped ring shape section 71 in addition to the step section 61. In other words, watertightness in a complicated-shaped boundary can be secured by the O-shaped ring shape section 71 not exposed to the boundary considering graspability, operability, and the like. On the other hand, a gap can be prevented from being formed in the boundary by the step section 61.

As explained above, it is possible to eliminate a deficiency that a hand or fingers grasping the grasping section 3 touches the elastic seal member 70 to adversely affect operability. When an external force is applied to the large diameter section outer circumferential surface 66 of the elastic seal member 70 and the large diameter section outer circumferential surface 66 is pressed, as explained above, the elastic seal member 70 is elastically deformed and the large diameter section inner surface 67 is in an abutted state with the second projection outer circumferential surface 11e. The large diameter section outer circumferential surface 66 is in a state in which the large diameter section outer circumferential surface 66 is not further pushed in by the external force. Thereafter, the external force applied to the large diameter section outer circumferential surface 66 is eliminated and the large diameter section inner surface 67 in a state of being elastically deformed and abutting the second projection outer circumferential surface 11e returns to an original state of the large diameter section inner surface 67. The large diameter section outer circumferential surface 66 returns to an original state of the large diameter section outer circumferential surface 66 without recessing with the external force. Therefore, it is possible to surely prevent a deficiency that the large diameter section outer circumferential surface 66 recesses with the external force.

Other action and effects are the same as those in the case of the elastic seal member 60 explained above. Explanation of the action and effects is omitted.

Note that, in the embodiment explained above, the abutting section or the like having the abutting surface is provided on the first exterior member side and the housing section or the like that houses the abutting section is provided on the second exterior member side. However, the abutting section or the like may be provided on the second exterior member side and the housing section or the like may be provided on the first exterior member side.

In the embodiment explained above, the exterior member and the elastic seal member are respectively configured as separate members. However, the elastic seal member may be integrally provided with, for example, the distal end side case, which is the exterior member, by insert molding.

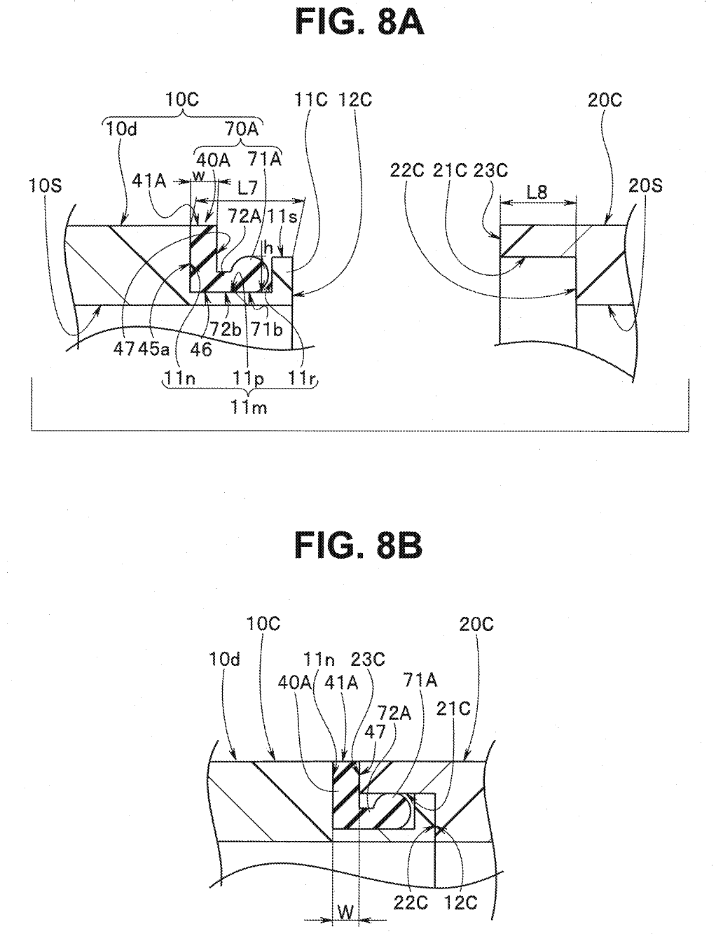

A distal end side case 10C shown in FIG. 8A includes a case section 10d and an elastic seal member 70A. The elastic seal member 70A is integrated with the case section 10d by insert molding.

The elastic seal member 70A includes an elastic seal section 40A, which is a ring shape section substantially the same as the first elastic seal member 40, a connecting shape section 72A, and an O-shaped ring shape section 71A. On the other hand, an abutting section 11C including a circumferential groove 11m is provided in the case section 10d. The circumferential groove 11m includes a circumferential groove distal end side side surface 11n, which is a first boundary surface, a circumferential groove bottom surface 11p, and a circumferential groove proximal end side side surface 11r.

In the present embodiment, a side surface 45a side of the elastic seal section 40A adheres and is fixed to the circumferential groove distal end side side surface 11n. An inner surface 46 side of the elastic seal section 40A, an inner surface 72b side of the connecting shape section 72A, and an inner surface 71b side of the O-shaped ring shape section 71A adhere to and are fixed to the circumferential groove bottom surface 11p.

The O-shaped ring shape section 71A has a substantially spherical shape. Height h of the O-shaped ring shape section 71A is set to further project, by a predetermined amount, than a proximal end side plane 11s orthogonal to the circumferential groove proximal end side side surface 11r configuring the circumferential groove 11m.

A sign 41A indicates an elastic seal section outer circumferential surface, which is one surface functioning as a case body exterior of the distal end side case 10C. The elastic seal section outer circumferential surface 41A is provided to be flush or substantially flush with an outer circumferential surface of the case section 10d in advance.

On the other hand, a center portion case 20C includes a housing section 21C. The connecting shape section 72A and the O-shaped ring shape section 71 of the elastic seal member 70A and a distal end side portion of the abutting section 11C are housed in the housing section 21C.

A bottom surface 22C of the housing section 21C is formed to recess from a distal end side boundary surface 23C, which is a second boundary surface, by a predetermined eighth distance L8. The bottom surface 22C is a contact surface with which an abutting surface 12C is in contact. An inner circumferential surface of the housing section 21C is an O-shaped ring contact surface that is in contact with an outer surface of the O-shaped ring shape section 71A.

In a state in which the abutting section 11C is housed in the housing section 21C and the abutting surface 12C is in contact with the bottom surface 22C, the O-shaped ring shape section 71A is set such that the O-shaped ring shape section 71 is crushed by a predetermined amount and elastically deformed and adheres to the inner circumferential surface of the housing section 21C and can secure watertightness.

In a state in which the abutting surface 12C is in contact with the bottom surface 22C, a distance between the circumferential groove distal end side side surface 11n and the distal end side boundary surface 23C, which is the width W of the boundary, is L7-L8. The distance between the circumferential groove distal end side side surface 11n and the distal end side boundary surface 23C is set smaller than the width w of the elastic seal section 40A.

In other words, the distance and the width w are set in a relation of L7-L8<w.

Therefore, the elastic seal section 40A having the width w is crushed by a predetermined crush amount and elastically deformed and adheres to at least prevent a gap from being formed in a state in which the abutting surface 12C is in contact with the bottom surface 22C.

Assembly of the distal end side case 10C and the center portion case 20C is explained.

In the present embodiment, the abutting section 11C of the distal end side case 10C, in which the elastic seal member 70A is provided, shown in FIG. 8A is engaged in the housing section 21C of the center portion case 20C and moved in a direction in which the elastic seal member 70A is held between the distal end side case 10C and the center portion case 20C. As a result, an outer circumferential surface of the O-shaped ring shape section 71A comes into contact with an inner circumferential surface of the housing section 21C. The distal end side boundary surface 23C moves toward the seal section end face 47 while crushing the O-shaped ring shape section 71A.

Thereafter, as shown in FIG. 8B, the distal end side boundary surface 23A comes into contact with the seal section end face 47, the bottom surface 22C comes into contact with the abutting surface 12C, and the abutting section 11C is housed in the housing section 21C in a predetermined state. The assembly of the distal end side case 10C and the center portion case 20C is completed.

In this way, the elastic seal member 70A adheres and is fixed to the circumferential groove distal end side side surface 11n and the circumferential groove bottom surface 11p of the abutting section 11C of the distal end side case 10C. Consequently, attachment work for attaching the elastic seal member 70A to the abutting section is unnecessary. Workability of assembly can be improved by reducing one watertight part.

In an assembly completed state, the O-shaped ring shape section 71A is elastically deformed and disposed to adhere to the inner circumferential surface of the housing section 21C and can surely retain watertightness of the distal end side case 10C and the center portion case 20C.

In addition, the distal end side boundary surface 23C comes into contact with the seal section end face 47 of the elastic seal section 40A of the elastic seal member 70A and crushes the elastic seal section 40A by a predetermined crush amount and is disposed to adhere to the elastic seal section 40A. The elastic seal section outer circumferential surface 41A does not further project than an outer circumferential surface of the distal end side case 10C and an outer circumferential surface of the center portion case 20B and is maintained flush or substantially flush with the outer circumferential surfaces. The same action and effects as those in the embodiment explained above can be obtained.

When an external force is applied to the elastic seal section outer circumferential surface 41A of the elastic seal member 70A, the external force is transmitted from the elastic seal section 40A to the connecting shape section 72A and the connecting shape section 72A is elastically deformed. Consequently, the connecting shape section 72A is restored to an original state of the connecting shape section 72A when the external force applied to the elastic seal section outer circumferential surface 41A is eliminated. The same action and effects as those in the embodiment explained above can be obtained.

Other action and effects are the same as those in the embodiment explained above.

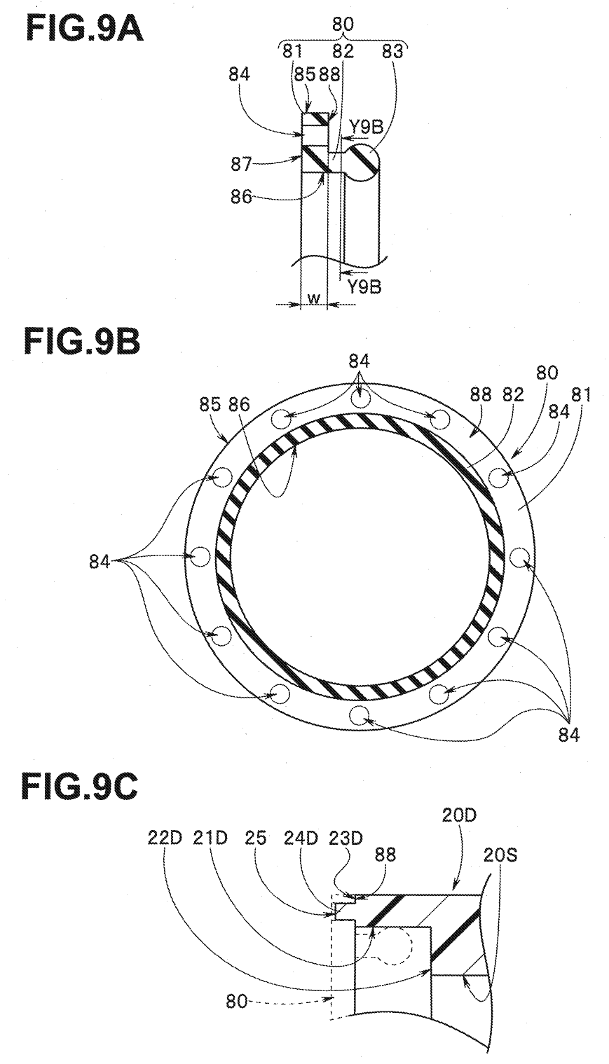

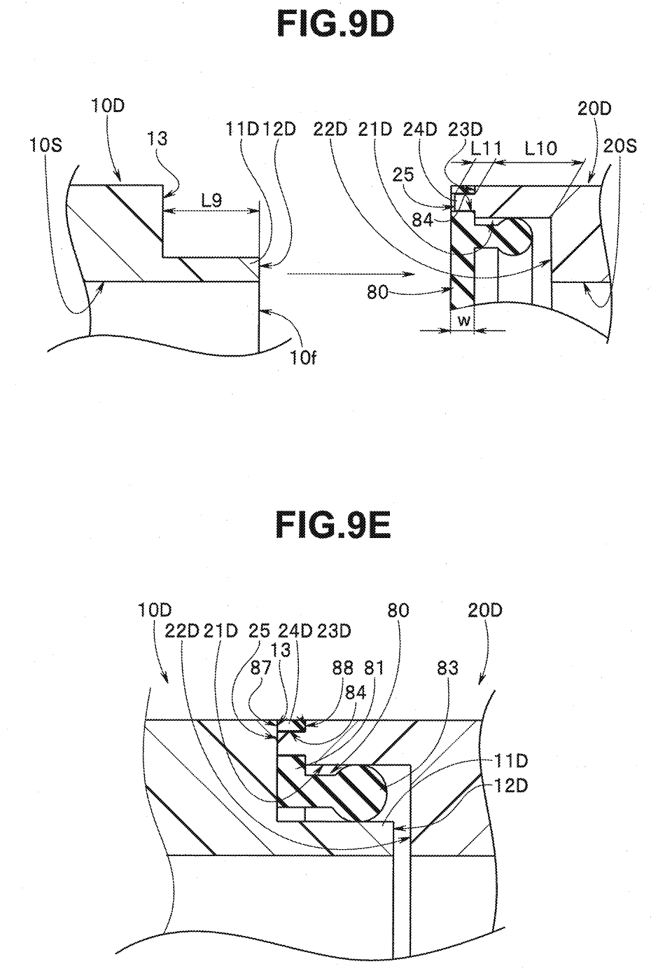

In the embodiment explained above, in order to eliminate a deficiency that an external force is applied to an outer circumferential surface of an elastic seal member exposed between boundary surfaces, whereby the outer circumferential surface is deformed, for example, the exposed two-step-shaped elastic seal member is formed as shown in FIG. 6A. However, an elastic seal member may be configured as shown in FIG. 9A and FIG. 9B and an exterior member, in which the elastic seal member is disposed, may be configured as shown in FIG. 9C to achieve elimination of the deficiency explained above.

As shown in FIG. 9A and FIG. 9B, an elastic seal member 80 includes a ring shape section 81, a connecting section 82, and an O-shaped ring shape section 83. In the ring shape section 81, a plurality of through-holes 84 are formed, for example, at equal intervals in a circumferential direction. The connecting section 82 integrally couples the ring shape section 81 and the O-shaped ring shape section 83.

A sign 85 indicates a ring outer circumferential surface, a sign 86 indicates a ring inner circumferential surface, a sign 87 indicates a boundary surface contact surface, and a sign 88 indicates a boundary surface adhering surface. The boundary surface contact surface 87 is in contact with the first exterior member boundary surface 13 shown in FIG. 9D. The boundary surface adhering surface 88 is disposed in contact with a distal end side boundary surface 23D. The boundary surface contact surface 87 is formed in a surface shape substantially coinciding with a surface shape of the first exterior member boundary surface 13. The boundary surface adhering surface 88 is formed in a surface shape substantially coinciding with the surface shape of the distal end side boundary surface 23D.

As shown in FIG. 9C, a center portion case 20D includes a housing section 21D. A projection 11D shown in FIG. 9D is housed in the housing section 21D. The projection 11D projects from the first exterior member boundary surface 13 by a predetermined ninth distance L9.

A bottom surface 22D of the housing section 21D is formed to recess from the distal end side boundary surface 23D by a predetermined amount. In the present embodiment, a distance from the distal end side boundary surface 23D to the bottom surface 22D is set to a predetermined tenth distance L10. A projection end face 12D of the projection 11D is housed in the housing section 21D without being in contact with the bottom surface 22D.

A sign 24D indicates an abutting section. The abutting section 24D has, for example, a columnar shape. The abutting section 24D is provided to correspond to a plurality of through-holes 84 formed in the elastic seal member 80. The abutting section 24D projects from the distal end side boundary surface 23D, which is a first boundary surface, by a predetermined amount. More specifically, a distance from the distal end side boundary surface 23D to an abutting surface 25 of the abutting section 24D is set to a predetermined eleventh distance L11.

Therefore, the distances are in a relation of L9<L10+L11.

Note that the eleventh distance L11 is set smaller than the width w of the ring shape section 81 in advance. An outer diameter of the abutting section 24D is set smaller than an inner diameter of the through-holes 84 in advance.

The elastic seal member 80 is disposed to be in contact with the distal end side boundary surface 23D in advance as indicated by a broken line in FIG. 9C. In this disposition state, the abutting section 24D is located in the through-holes 84.

A configuration of a distal end side case 10D and assembly of the distal end side case 10D and a center portion case 20D are explained with reference to FIG. 9D and FIG. 9E.

As shown in FIG. 9D, a projection 11D is provided on a proximal end opening 10f side of the distal end side case 10D. A projection end face 12D of the projection 11D projects from the first exterior member boundary surface 13 by the predetermined ninth distance L9 explained above.

In the present embodiment, the projection 11D is a seal member engaging section. Therefore, an inward side of the O-shaped ring shape section 83 of the elastic seal member 80 is disposed on an outer circumferential surface of the projection 11D. After the end face 12D of the projection 11D passes through an inner hole of the O-shaped ring shape section 83, the projection end face 12D is located in the housing section 21D without being in contact with the bottom surface 22D. The ring shape section 81 having the width w is set such that, in this disposition state, the abutting surface 25 comes into contact with the first exterior member boundary surface 13, whereby the ring shape section 81 is crushed by a predetermined crush amount and elastically deformed. In other words, in the present embodiment, the first exterior member boundary surface 13 is a contact surface that is in contact with the abutting surface 25. In a contact state, a gap is provided between the projection end face 12D and the bottom surface 22D.

When the distal end side case 10D and the center portion case 20D are assembled, the elastic seal member 80 is attached to the center portion case 20D as shown in FIG. 9D. At this time, the abutting section 24D is disposed in the through-holes 84 of the elastic seal member 80. The boundary surface adhering surface 88 is disposed on the distal end side boundary surface 23D.

The projection 11D of the distal end side case 10D is engaged in the ring inner circumferential surface 86 of the ring shape section 81 disposed in the housing section 21D of the center portion case 20D, to which the elastic seal member 80 is attached, to move the projection end face 12D of the projection 11D toward the bottom surface 22D of the housing section 21D in a direction in which the elastic seal member 80 is held between the distal end side case 10D and the center portion case 20D.

Then, an inward surface of the O-shaped ring shape section 83 of the elastic seal member 80 comes into contact with the outer circumferential surface of the projection 11D. The projection end face 12D moves toward the bottom surface 22D while crushing the O-shaped ring shape section 83.

Thereafter, as shown in FIG. 9E, the first exterior member boundary surface 13 comes into contact with the boundary surface contact surface 87 of the ring shape section 81 and crushes the ring shape section 81. Thereafter, the first exterior member boundary surface 13 and the abutting surface 25 come into contact with each other.

As a result, the boundary surface adhering surface 88 is disposed to adhere to the distal end side boundary surface 23D. The projection 11D is housed in the housing section 21D. The assembly of the distal end side case 10D and the center portion case 20D is completed.

At this time, the ring shape section 81 is deformed such that an inner circumferential surface of the through-holes 84 adheres to an outer circumferential surface of the abutting section 24D without the ring outer circumferential surface 85 being deformed to an outward side by elastic deformation.

In this way, the O-shaped ring shape section 83 is elastically deformed and disposed to adhere to an inner circumferential surface of the housing section 21D and the outer circumferential surface of the projection 11D and can surely retain watertightness of the distal end side case 10D and the center portion case 20D.

In addition, the boundary surface contact surface 87 of the ring shape section 81 of the elastic seal member 80 comes into contact with the first exterior member boundary surface 13 and crushes the ring shape section 81 by a predetermined crush amount and is disposed to adhere to the ring shape section 81. The ring outer circumferential surface 85 of the ring shape section 81 does not further project than an outer circumferential surface of the distal end side case 10D and an outer circumferential surface of the center portion case 20B and is maintained flush or substantially flush with the outer circumferential surfaces. The same action and effects as those in the embodiment explained above can be obtained.

When an external force is applied to the ring outer circumferential surface 85 of the ring shape section 81 of the elastic seal member 80, since the abutting section 24D is disposed in the through-holes 84 formed in the ring shape section 81, a part of the external force applied to the ring outer circumferential surface 85 of the ring shape section 81 acts on the abutting section 24D and the elastic deformation of the ring shape section 81 is reduced. When the external force applied to the ring outer circumferential surface 85 is eliminated, the ring outer circumferential surface 85 is restored to an original state of the ring outer circumferential surface 85. The same action and effects as those in the embodiment explained above can be obtained.

Note that, in the present embodiment, the abutting section 24D is formed in a columnar shape. However, the abutting section 24D may be formed in a square pillar shape. Other action and effects are the same as those in the embodiment explained above.

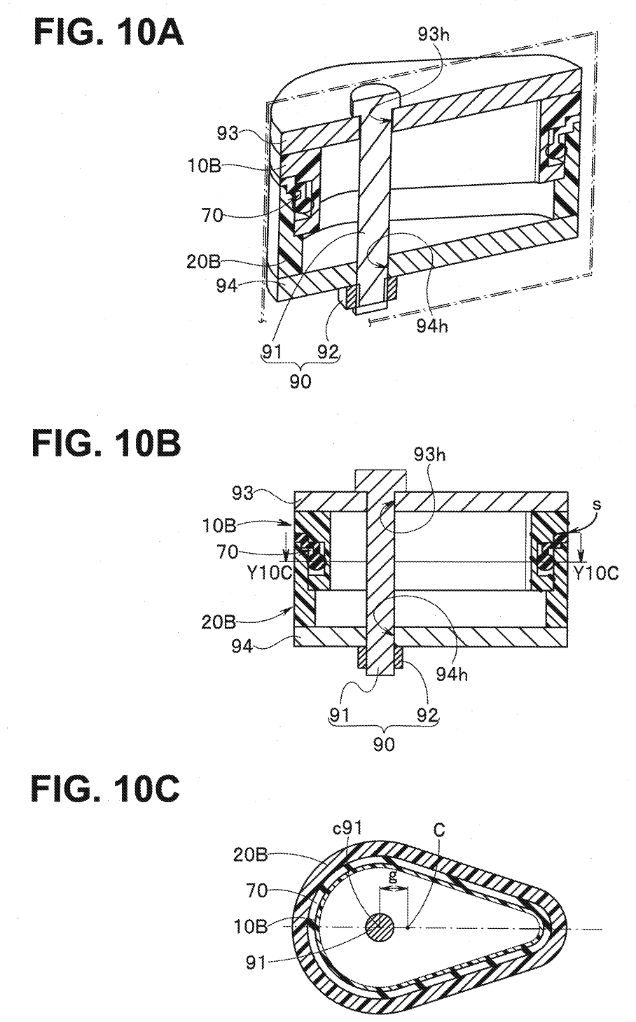

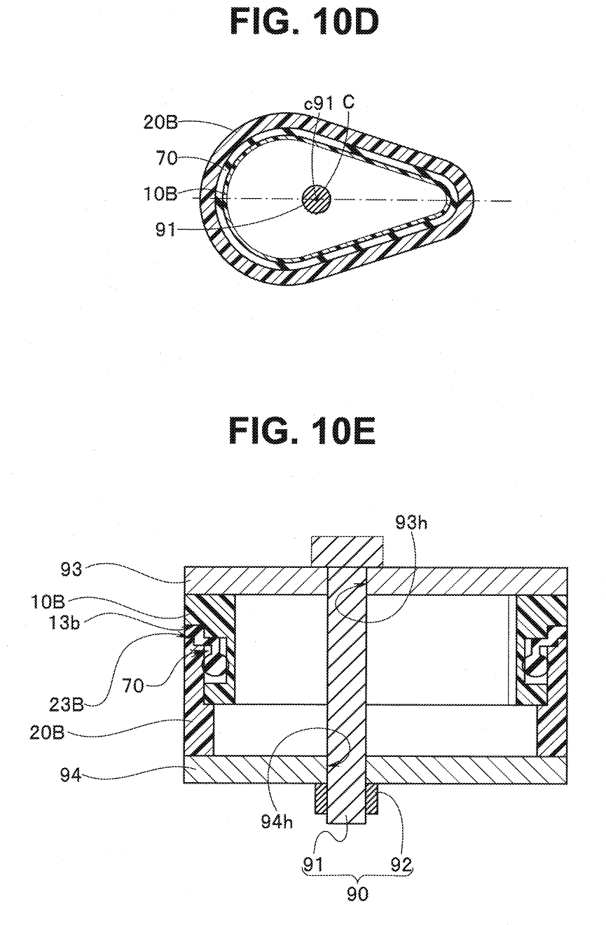

A fixing method after assembly is explained with reference to FIG. 10A to FIG. 10E.

As shown in FIG. 10A and FIG. 10B, the distal end side case 10B and the center portion case 20B after assembly completion are firmly fixed by, for example, a bolt 91 and a nut 92, which form a fastening member 90. A sign 93 and a sign 94 indicate a pair of supporting members.

After assembly completion, a first supporting member 93 is disposed on the distal end side case 10B side and a second supporting member 94 is disposed on the center portion case 20B side. The distal end side case 10B, the elastic seal member 70, and the center portion case 20B are integrally fastened by the bolt 91 and the nut 92.

A first bolt passage hole 93h is formed in the first supporting member 93. A second bolt passage hole 94h is formed in the second supporting member 94. Therefore, for example, the bolt 91 is disposed to pass through the first bolt passage hole 93h on the first supporting member 93 side to the second bolt passage hole 94h on the second supporting member 94 side. The nut 92 is screwed with a distal end portion of the bolt 91 projecting from the second supporting member 94.

When a center axis c91 of the bolt 91 in a fastened state positionally deviates from a center of figure C of a contact surface by, for example, a distance g as shown in FIG. 10C, a holding force applied to the large diameter section 62 of the elastic seal member 70 disposed between the first outer side boundary surface 13b and the distal end side boundary surface 23B is likely to be ununiform because of a difference in a moment.

The holding force becoming ununiform causes a deficiency that a gap s is formed in a part where a holding force of the first outer side boundary surface 13b and the distal end side boundary surface 23 is smaller than a preset amount of force.

Accordingly, when the distal end side case 10B and the center portion case 20B having a complicated-shaped boundary are fastened considering graspability, operability, and the like, a center axis of the bolt 91 disposed in the first bolt passage hole 93h provided in the first supporting member 93 and the second bolt passage hole 94h provided in the second supporting member 94 is matched with the center of figure C of the contact surface as shown in FIG. 10D.

As a result, it is possible to elastically deform, with a uniform holding force having the preset amount of force, the large diameter section 62 of the elastic seal member 70 disposed between the first outer side boundary surface 13b and the distal end side boundary surface 23B as shown in FIG. 10E.

Note that, although not illustrated, shapes of the first outer side boundary surface 13b and the distal end side boundary surface 23B may be adjusted to match the center axis of the bolt 91 with the center of figure C of the contact surface without changing the first bolt passage hole 93h provided in the first supporting member 93 and the second bolt passage hole 94h provided in the second supporting member 94.

Note that the insertion apparatus is not limited to the endoscope 1 including the insertion section 2 configured to be inserted into the subject and may be a treatment instrument or the like including an insertion section configured to be inserted into the subject.

The present invention is not limited only to the embodiment explained above and can be variously modified and implemented in a range not departing from the gist of the invention.

According to the present invention, it is possible to realize a case body and an insertion apparatus including the case body in which control of a crushing amount of an elastic seal member is easy, a gap is not formed in a boundary between exterior members provided adjacent to each other, and the elastic seal member is disposed not to project or recess with respect to an exterior member outer surface to prevent deterioration in durability while retaining watertightness of an internal space.

What is claimed is:

1. A case body comprising:
a first exterior member configuring a part of a case body exterior;
a second exterior member disposed in a position adjacent to the first exterior member and configuring the case body exterior in conjunction with the first exterior member;
an elastic seal member having predetermined elasticity disposed to be exposed as the case body exterior in a boundary between the first exterior member and the second exterior member provided adjacent to each other; and
an abutting member configured to set a crush amount of the elastic seal member, the abutting member including an abutting surface provided in one of the first exterior member and the second exterior member and coming into contact with another exterior member, wherein
the abutting member is a projection provided in the first exterior member and projecting with respect to a first boundary surface configuring one side of the boundary,
the second exterior member includes a recess formed to recess with respect to a second boundary surface configuring another side of the boundary, the projection being housed in the recess,
the elastic seal member is held between the first boundary surface and the second boundary surface in a state in which the projection and the recess are engaged, and
the elastic seal member is formed in a stepped shape including a large diameter section formed in a ring shape and a small diameter section formed in a ring shape smaller in diameter than the large diameter section, and an outer circumferential surface of the large diameter section is disposed to be exposed as the case body exterior in the boundary between the first exterior member and the second exterior member.

2. A case body comprising:
a first exterior member configuring a part of a case body exterior;
a second exterior member disposed in a position adjacent to the first exterior member and configuring the case body exterior in conjunction with the first exterior member;
an elastic seal member having predetermined elasticity, the elastic seal member including a large diameter section formed in a ring shape and a small diameter section formed in a ring shape smaller in diameter than the large diameter section and being disposed to be exposed as the case body exterior in a boundary between the first exterior member and the second exterior member provided adjacent to each other;
an abutting member configured to set a crush amount of the elastic seal member, the abutting member including an abutting surface provided in one of the first exterior member and the second exterior member and coming into contact with another exterior member;
a watertightness retaining member disposed to be further separated from the case body exterior than the elastic seal member in the boundary between the first exterior member and the second exterior member and configured to retain watertightness; and
a coupling ring formed to project from the small diameter section and coupled to the watertightness retaining member.

3. The case body according to claim 1, wherein the abutting member sets a crushing amount of the elastic seal member such that the elastic seal member smoothly forms the case body exterior in conjunction with the first exterior member and the second exterior member.

4. The case body according to claim 2, wherein the abutting member sets a crushing amount of the elastic seal member such that the elastic seal member smoothly forms the case body exterior in conjunction with the first exterior member and the second exterior member.

5. The case body according to claim 1, wherein the elastic seal member includes a rectangular-shaped cross section in a direction substantially orthogonal to a boundary surface between the first exterior member and the second exterior member.

6. The case body according to claim 2, wherein the elastic seal member includes a rectangular-shaped cross section in a direction substantially orthogonal to a boundary surface between the first exterior member and the second exterior member.

7. The case body according to claim 1, wherein the elastic seal member is integrally provided in one of the first exterior member or the second exterior member.

8. The case body according to claim 2, wherein the elastic seal member is integrally provided in one of the first exterior member or the second exterior member.

9. The case body according to claim 1, wherein width of the elastic seal member is set such that the elastic seal member is held between the first boundary surface and the second boundary surface and elastically deformed by a predetermined amount in a state in which the projection and the recess are engaged.

10. The case body according to claim 1, further comprising a watertightness retaining member disposed to be further separated from the case body exterior than the elastic seal member in the boundary between the first exterior member and the second exterior member and configured to retain watertightness.

11. The case body according to claim 2, wherein
the abutting member is a projection provided in the first exterior member and projecting with respect to a first boundary surface configuring one side of the boundary,
the second exterior member includes a recess formed to recess with respect to a second boundary surface configuring another side of the boundary, the protrusion being housed in the recess, and
width of the elastic seal member is set such that the elastic seal member is held between the first boundary surface and the second boundary surface and elastically deformed by a predetermined amount in a state in which the projection and the recess are engaged.

12. The case body according to claim 2, wherein the elastic seal member has a stepped shape including a large diameter section formed in a ring shape and a small diameter section formed in a ring shape smaller in diameter than the large diameter section, and an outer circumferential surface of the large diameter section is disposed to be exposed as the case body exterior in the boundary between the first exterior member and the second exterior member.

13. The case body according to claim 2, wherein the elastic seal member is formed in a ring shape and includes a plurality of through-holes in a circumferential direction.

14. The case body according to claim 13, wherein an abutting section projecting from one surface configuring a boundary surface between the first exterior member and the second exterior member is disposed in the through-holes.

15. An insertion apparatus comprising:
an insertion section configured to be inserted into a subject; and
a case body including:
a first exterior member provided on a distal end side of the insertion section and configuring a part of an internal space and a part of a case body exterior;
a second exterior member disposed in a position adjacent to the first exterior member and configuring the case body exterior in conjunction with the first exterior member;
an elastic seal member having predetermined elasticity disposed to be exposed as the case body exterior in a boundary between the first exterior member and the second exterior member provided adjacent to each other; and
an abutting member configured to set a crush amount of the elastic seal member, the abutting member including an abutting surface provided in one of the first exterior member and the second exterior member and coming into contact with another exterior member, wherein
the abutting member is a projection provided in the first exterior member and projecting with respect to a first boundary surface configuring one side of the boundary,
the second exterior member includes a recess formed to recess with respect to a second boundary surface configuring another side of the boundary, the projection being housed in the recess,
the elastic seal member is held between the first boundary surface and the second boundary surface in a state in which the projection and the recess are engaged, and
the elastic seal member is formed in a stepped shape including a large diameter section formed in a ring shape and a small diameter section formed in a ring shape smaller in diameter than the large diameter section, and an outer circumferential surface of the large diameter section is disposed to be exposed as the case body exterior in the boundary between the first exterior member and the second exterior member.

16. An insertion apparatus comprising:
an insertion section configured to be inserted into a subject; and
a case body including:
a first exterior member provided on a distal end side of the insertion section and configuring a part of an internal space and a part of a case body exterior;
a second exterior member disposed in a position adjacent to the first exterior member and configuring the case body exterior in conjunction with the first exterior member;
an elastic seal member having predetermined elasticity, the elastic seal member including a large diameter section formed in a ring shape and a small diameter section formed in a ring shape smaller in diameter than the large diameter section and being disposed to be exposed as the case body exterior in a boundary between the first exterior member and the second exterior member provided adjacent to each other;
an abutting member configured to set a crush amount of the elastic seal member, the abutting member including an abutting surface provided in one of the first exterior member and the second exterior member and coming into contact with another exterior member;
a watertightness retaining member disposed to be further separated from the case body exterior than the elastic seal member in the boundary between the first exterior member and the second exterior member and configured to retain watertightness; and
a coupling ring formed to project from the small diameter section and coupled to the watertightness retaining member.
